(19) United States
(12) Patent Application Publication
(10) Pub. No.: US 2022/0041638 A1
(43) Pub. Date: Feb. 10, 2022

(54) METHOD FOR SEPARATING BIOMASS FROM A SOLUTION COMPRISING BIOMASS AND AT LEAST ONE OLIGOSACCARIDE

(71) Applicant: BASF SE, Ludwigshafen am Rhein (DE)

(72) Inventors: Jacek MALISZ, Ludwigshafen am Rhein (DE); Daniel SEIBERT-LUDWIG, Ludwigshafen am Rhein (DE); Peter OEDMAN, Saint Joseph, MO (US); Michael PUHL, Ludwigshafen am Rhein (DE)

(21) Appl. No.: 17/414,703

(22) PCT Filed: Dec. 17, 2019
(86) PCT No.: PCT/EP2019/085479

§ 371 Date: Jun. 16, 2021

(30) Foreign Application Priority Data
Dec. 19, 2018 (EP) 18214004.6

(51) Int. Cl. C07H 1/08; C07H 3/06; B01D 61/14; B01D 71/02; B01D 69/02; B01D 71/68; B01D 61/16

(52) U.S. Cl. CPC C07H 1/08; B01D 2311/06; B01D 61/147; B01D 61/145; B01D 71/025; B01D 69/02; B01D 71/68; B01D 61/142; B01D 61/16; B01D 2315/10; B01D 2325/02; B01D 2325/20; B01D 2311/2626; B01D 2311/18; B01D 2311/04; C07H 3/06

(57) ABSTRACT

The present invention relates to a method for separating biomass from a solution comprising biomass and at least one oligosaccharide.comprising providing the solution comprising biomass and oligosaccharides.lowering the pH value of the solution below 7 by adding at least one acid to the solution comprising biomass and the at least one oligosaccharide. adding an adsorbing agent to the solution comprising biomass and oligosaccharides. and carrying out first membrane filtration so as to separate the biomass from the solution comprising the at least one oligosaccharide.

### TECHNICAL FIELD

The present invention relates to a method for separating biomass from a solution comprising biomass and at least one oligosaccharide.

### BACKGROUND

Human milk oligosaccharides (HMOs) are the third most abundant solid component of human milk after lactose and lipids. The concentrations of different HMOs and their total amount in human milk vary within the lactation phase and between individuals, which is believed to be partially based on genetic background. Importantly, however, HMOs are not found in comparable abundances in other natural sources, like cow, sheep, or goat milk. Several beneficial effects of HMOs on infants have been shown or suggested, including selective enhancement of bifidobacterial growth, anti-adhesive effects on pathogens and glycome-altering effects on intestinal epithelial cells. The trisaccharide 2′-fucosyllactose (2′-FL) is one of the most abundant oligosaccharides found in human milk. Due to its prebiotic and anti-infective properties, 2′-FL is discussed as nutritional additive for infant formula. Moreover, infants' nutrition containing 2′-FL is associated with lower rates of diarrhea, making 2′-FL a potential nutritional supplement and therapeutic agent, if it were available in sufficient amounts and at a reasonable price.

Formerly, 2′-FL has been obtained via extraction from human milk or chemical synthesis, but the limited availability of human milk or the necessity of side group protection and deprotection in chemical synthesis, respectively, set limits to supply and cost efficiency. Thus, alternative sources of 2′-FL became of interest. Besides chemical synthesis and extraction from human milk, 2′-FL can be produced enzymatically in vitro and in vivo. The most promising approach for a large-scale formation of 2′-FL is the whole cell biosynthesis in Escherichia coli by intracellular synthesis of GDP-L-fucose and subsequent fucosylation of lactose with an appropriate α1,2-fucosyltransferase.

Thus, HMOs may be produced by means of fermentation providing a solution comprising biomass and at least one oligosaccharide, preferably 2′-FL. Such a solution may also be called fermentation broth.

Biomass separation from the fermentation broth from the HMO process is the first downstream processing step in the production of HMO. The state-of-the-art technology for this step is centrifugation and or filter press, sometimes with the use of flocculants. However, microfiltration can also be employed and has several advantages in comparison to other separation technologies. To enable a genetically modified organism free product solution, microfiltration is the best option because it can completely retain all non-dissolved solids including genetically modified microorganisms.

### SUMMARY

Membrane filtrations are often used to separate smaller molecules from larger ones in a solution. One example for oligosaccharide containing solutions is disclosed in the Chinese patent application published as CN 100 549 019, a patent application disclosing a method for preparing high-purity xylooligosaccharide from straw by using enzyme and membrane technology. Another example is disclosed in EP 2 896 628, a patent application disclosing a membrane filtration of oligosaccharide containing fermentation broth followed by performing further process steps including addition of activated carbon to the filtrate.

The separation of the biomass after fermentative production of HMO is usually done at a pH value of 7 by means of an initial centrifugation or filter press and further centrifugations. Sometimes polymeric membranes are used instead.

When membranes are used, however, the membrane performance is rather low and the permeate contains a lot of proteins and color components, which have to be removed in the following steps leading to an elaborate downstream process, high product yield losses and some quality problems.

Typically, after these initial steps of biomass separation from fermentation broths the next step carried out is an ultrafiltration completed typically with 10 kDa polyethersulfone membranes, yet not all proteins and polysaccharides can be separated by this. The ultrafiltration permeate is hence set to an active carbon column to decolorize the solution and achieve an APHA value of below 1000. The decolorization in the active carbon column is a rather tedious process and it is often necessary to use around 14% weight/weight of active carbon in relation to the initial amount of fermentation broth. This step leads to high product losses and necessitates huge active carbon columns.

It was therefore an object of the invention to avoid the abovementioned disadvantages. In particular, a method should be provided that is suitable to enhance the performance of separating biomass from a solution comprising biomass and at least one oligosaccharide and to reduce the amount of proteins in and the color of the filtration permeate.

According to the present invention, this object is solved by a method for separating biomass from a solution comprising biomass and at least one oligosaccharide, comprising:
providing the solution comprising biomass and oligosaccharides,
lowering the pH value of the solution below 7, preferably below pH 5.5 or less by adding at least one acid to the solution comprising biomass and the at least one oligosaccharide,
adding an adsorbing agent to the solution comprising biomass and oligosaccharides, and
carrying out a membrane filtration also called herein the first membrane filtration and typically being a microfiltration or ultrafiltration so as to separate the biomass from the solution comprising the at least one oligosaccharide. Preferably, the sequence of method steps is the one given in the previous sentence.

According to the method of the present invention, it was surprisingly found, that the membrane performance can be significantly increased, and removal of proteins can be significantly improved when the pH value of the solution is lowered below 7. Further, it was found that membrane performance increases further and the color of the permeate can be significantly reduced to values below the required specification when an adsorbing agent is added to the solution before any membrane filtration. Also advantageously, the needed amount of adsorbing agent like active carbon is much lower as compared to the known methods, and also the required time for decolorization is much shorter than in known methods, when the membrane filtration is done after the pH value has been set to the desired target value below pH 7 and at least on adsorbing agent has been added.

Preferably, the adsorbing agent is active carbon. Active carbon, also known as activated carbon or activated charcoal, is a preferred adsorbing agent as it is of low cost, available in large quantities, easy to handle and safe to food.

It is beneficial to the methods of the invention that the pH value of the solution comprising biomass and one or more oligosaccharide, one or more disaccharide and/or one or more monosaccharide is below pH 7.0 when the first membrane filtration is performed, and more preferably when the adsorbing agent is added. Hence, since pH values of fermentation broth are typically at or above pH 7.0, the pH value is lowered by the addition of at least one acid as needed to achieve the target pH value. In case the pH value of the solution comprising biomass and one or more oligosaccharide, one or more disaccharide and/or one or more monosaccharide is already below pH 7.0 at the start, at least one acid may be used for setting the pH value stably below pH7.0 as needed. Also preferably, the pH value of the solution is set to a pH value of 5.5 or below, before any membrane filtration is started. Preferably the pH value is lowered to a target pH value in the range of 3.0 to 5.5, more preferably the range of 3.5 to 5, wherein the ranges given include the given numbers. In an even more preferred embodiment, the pH value of the solution is set to pH 3.5 or above, but not higher than pH 4.5 and most preferably the pH value is set to a value in the range of and including 4.0 to 4.5. To this end, at least one acid is added to the solution. Said at least one acid is, more preferably, an acid selected from the group consisting of H₂SO₄, H₃PO₄, HCl, HNO₃ and CH₃CO₂H. Basically, any acid may be used. Nevertheless, these acids are usually easy to handle.

Said adsorbing agent, preferably active carbon, is typically added in an amount in the range of 0.25% to 3% by weight, preferably in the range of 0.5% to 2.5% by weight and more preferably in the range of 0.75% by weight to 2.2% by weight and even more preferably in the range of 1.0% to 2.0% by weight, wherein the percentage values are on a weight of adsorbing agent per weight of solution basis. Thus, a rather small amount of said adsorbing agent, preferably active carbon, is sufficient to reduce the color number below the upper bound specification, which is preferably 1000 APHA. This allows for significant reduction of active carbon consumption as well as for significant reduction of product losses in comparison to the active carbon column. In one embodiment one or more adsorbing agents are added in an amount suitable to bind—in increasing order of preference—at least 50%, 55%, 60%, 65%, 70%, 75%, 80%, 90%, 92%, 94%, 95% or more of the color components and/or the protein in the starting solution comprising biomass and/or polysaccharides and/or proteins and/or nucleic acids like DNA or RNA that may be present. Further, said adsorbing agent, preferably active carbon, is typically added as a powder having a particle size distribution with a diameter d50 in the range of 2 μm to 25 μm, preferably in the range of 3 μm to 20 μm and more preferably in the range of 3 μm to 7 μm, and even more preferably in the range of 5 μm to 7 μm. The d50 value is determined with standard procedures. Particle sizes in this size range reduce the risk of abrasion of the membrane. Moreover, said adsorbing agent, preferably active carbon, is yet preferably added as a suspension of the powder in water. This facilitates handling of the adsorbing agent as the suspension of the powered may better mix with the suspension comprising biomass and the oligosaccharide. The adding said adsorbing agent, preferably active carbon, to the solution is, typically, carried out after adding the at least one acid to the solution. Unexpectedly, the color reduction and protein reduction are much better, when the pH value is adjusted first and then the adsorbing agent or at least the majority of the adsorbing agent is added subsequently. It is possible to add said adsorbing agent, preferably active carbon, to the fermentation broth before adding the at least one acid to the solution.

In another variant, the pH value of the solution is lowered to 5.5, more preferably to 5.0 and even more preferably to 4.5 by the addition of at least one of the suitable acids, and then adsorbing agent, preferable active carbon, and further acid is added until the desired final pH value is achieved.

Also, some of the adsorbing agent may be added before any acid is added to lower the pH value, followed by the addition of more adsorbing agent after the pH value has been set to the target value below pH 7.0.

Preferably, said solution comprising biomass and oligosaccharides, typically a fermentation broth, is obtained by cultivation of one or more types of cells, preferably bacteria or yeast, more preferably bacteria, even more preferably genetically modified Escherichia coli, in a cultivation medium, preferably a cultivation medium comprising at least one carbon source, at least one nitrogen source and inorganic nutrients. Thus, sufficient amounts of said oligosaccharide may be produced with cost efficient methods.

Preferably, providing the solution comprising biomass and at least one oligosaccharide includes preparing said solution by means of microbial fermentation. Thus, sufficient amounts of said oligosaccharide may be produced with cost efficient methods.

Said microfiltration or ultrafiltration of the first membrane filtration step is typically carried out as cross-flow microfiltration or cross-flow ultrafiltration. Thus, the filtration efficiency may be enhanced. Said cross-flow microfiltration or cross-flow ultrafiltration includes a cross-flow speed above 0.2 m/s, preferably in the range of 0.5 m/s to 6.0 m/s, more preferably in the range of 2.0 m/s to 5.5 m/s and even more preferably in the range of 2.8 m/s to 4.5 m/s, and most preferably in the range of 3.0 m/s to 4.0 m/s if ceramic mono- and multi-channel elements are used. In another embodiment, the cross-flow speed is equal to or below 3.0 m/s. In case that a polymeric membrane is used for the first membrane filtration, cross-flow speeds of 2 m/s or less can be used; cross-flow speeds in the range of 0.5 m/s to 1.7 m/s are preferably used, but even cross-flow speeds of 0.5 m/s or less may be used. In another preferred embodiment, the cross-flow speed is not more than 1.7 m/s, 1.6 m/s, 1.5 m/s, 1.4 m/s, 1.3 m/s, 1.2 m/s, 1.1 m/s or 1.0 m/s if a polymeric membrane is used. Thus, the filtration speed may be optimized when compared to a filtration process without including a pH value adjustment and addition of an adsorbing agent. By doing so, wear and tear on and/or energy consumption of the membrane filtration equipment can be reduced by operating at lower cross-flow speed compared to previously known methods, while resulting in good separation.

Said first membrane filtration, preferably a microfiltration or ultrafiltration is, typically, carried out at a temperature of the solution in the range of 4° C. to 55° C., preferably in the range of 10° C. to 50° C. and more preferably in the range of 30° C. to 40° C. Thus, the temperature during said filtration step may be the same as during fermentation which further improves the membrane performance and decreases viscosity of the solution comprising biomass and oligosaccharide. Yet, the first membrane filtration is, also preferably, carried out by means of a ceramic microfiltration membrane or ceramic ultrafiltration membrane having a pore size in the range of 20 nm to 800 nm, preferably in the range of 40 nm to 500 nm and more preferably in the range of 50 nm to 200 nm. It is also possible to use multi-layered membranes that are engineered to have improved abrasion resistance, e.g. 400 nm and 200 nm and 50 nm pore size layers of Al₂O₃.Thus, sufficient amounts of proteins and polysaccharides may be removed in order to comply with the desired specification. Also typically, first membrane filtration is carried out by means of a polymeric microfiltration membrane or polymeric ultrafiltration membrane having a cut-off above or equal to 4 kDa, preferably in the range of 10 kDa to 200 nm, more preferably in the range of 50 kDa to 200 nm and even more preferably equal to or above 50 kDa. In another preferred embodiment the cut-off is 100 nm or less. Thus, sufficient amounts of proteins and polysaccharides may be removed in order to comply with the desired specification.

The polymeric material of the polymeric microfiltration membrane or polymeric ultrafiltration membrane is, preferably, at least one polymeric material selected from the group consisting of: polyethersulfone, polysulfone, polypropylene, polyvinylidene fluoride, polyacrylonitrile, polyvinylidene fluoride. Modified polymeric materials can also be used, for example hydrophilized polyethersulfone.

The ceramic material of the ceramic microfiltration membrane or ceramic ultrafiltration membrane is, preferably, at least one ceramic material selected from the group consisting of: TiO₂, ZrO₂, SiC and Al₂O₃.

The first membrane filtration, preferably microfiltration or ultrafiltration is, typically, carried out after a predetermined time after the adsorbing agent, preferably active carbon, has been added to the solution. This allows to provide an adsorption time during which color components are adsorbed. Said predetermined time is at least 2 min, preferably at least 10 min and more preferably at least 20 min. Thus, the adsorption of color components is rather quick.

The method may, preferably, further comprise carrying out a second or further membrane filtration, preferably an ultrafiltration, using the solution essentially free of biomass obtained by the microfiltration or ultrafiltration of the first membrane filtration and comprising one or more oligosaccharide, one or more disaccharides and/or one or more monosaccharides, preferably comprising the majority of these saccharides from the starting solution, e.g. the fermentation broth, that also comprised the biomass. Preferably, the second membrane filtration is done with the permeate of the first membrane filtration and with a membrane having a lower cut-off than the first membrane. Thus, an advantageous further processing of the permeate obtained by the first membrane filtration is realized. The second membrane filtration is, typically, an ultrafiltration carried out by means of an ultrafiltration membrane, preferably, at least partially made of a polymeric material, and having a cut-off in the range of 1 kDa to 10 kDa, preferably in the range of 2 kDa to 10 kDa and more preferably in the range of 4 kDa to 5 kDa.

The second membrane filtration may be performed with a ceramic membrane of 1 to 25 kDa cut-off. In a further embodiment it is preferable that the membrane is at least partially made of a polymeric material. Said polymeric material is, more preferably, at least one polymeric material selected from the group consisting of: polyethersulfone, polysulfone, polyacrylonitrile, cellulose acetate. Said second membrane filtration is, typically, carried out after adjusting the temperature of the solution to temperatures of below 20, preferably at a temperature of the solution being in the range of 4° C. to 15° C., preferably in the range 8° C. to 13° C. and more preferably in the range 8° C. to 12° C.

In a preferred embodiment, the first membrane filtration employed in the inventive methods includes two or preferably three steps as will be explained in further detail below. The first step includes a first diafiltration having a diafiltration factor (DF) .(amount of diafiltration water=starting amount of fermentation broth x diafiltration factor) ranging from 0.5 or less to 3 or above. Fore example, for 2′FL comprising solutions it was advantageous to have a DF of 0.5 while for other HMO molecules values of 3 proved to be better if a concentration step was to follow. During diafiltration, the amount of water or a suitable aqueous solution added is identical to the amount of permeate discharged. In a batch wise diafiltration, the volume in the feed vessel is thus kept constant. The second step includes concentrating of the fermentation broth preferably with a factor 2 or more by stopping the feed of diafiltration water and the level will decrease down to the target value (target value=volume or mass at the beginning of the fermentation broth/concentrating factor). Optionally, the subsequent third step includes a second diafiltration. By means of these three steps a lower dilution of the product within the permeate and an increased yield of ≥95% are realized. By increasing the factor of the second diafiltration, the yield may even be further increased.

The permeate then typically is the combination of all solutions passing through the membrane in these three steps. In a batch process each step produces a permeate fraction in a time-separated manner, that can be collected in one vessel for mixing, or processed separately. In a continuing process, each of the three steps produces a permeate fraction not in a time separated, and these fractions can be combined to form the permeate combined or treated separately if desired.

Optionally the first step of the first membrane filtration may be repeated one or more times, before the second step of concentration is done. Optionally, the second step may be performed, or it may be skipped if concentrating the solution is not desirable. This is useful when the fermentation broth has a high viscosity and or very high biomass content, for example.

Optionally the first step may be skipped and alternatively the second step is done without the first step, so that first a concentration of the fermentation broth is done while creating permeate, and then a diafiltration of the last step is done by feeding water or aqueous solutions to the solution comprising biomass and one or more oligosaccharide, disaccharide or monosaccharide.

Preferably, the at least one oligosaccharide comprises human milk oligosaccharide, preferably neutral or sialylated human milk oligosaccharide and more preferably Lacto-N-tetraose, Lacto-N-neotetraose, 3′-sialyllactose, 6′-sialyllactose and/or 2′-fucosyllactose, and even more preferably 2′-fucosyllactose, 6′-sialyllactose and/or Lacto-N-tetraose.

In one embodiment of the invention, the methods of the invention are applied for the separation of mono-and/or disaccharides from biomass from a solution containing mono-and/or disaccharides and biomass, for example for the separation of lactose, fucose, maltose or saccharose from biomass

A further embodiment is the inventive apparatus suitable to perform the methods of the invention.

Further features and embodiments of the invention will be disclosed in more detail in the subsequent description, particularly in conjunction with the dependent claims. Therein the respective features may be realized in an isolated fashion as well as in any arbitrary feasible combination, as a skilled person will realize. The embodiments are schematically depicted in the figures. Therein, identical reference numbers in these figures refer to identical elements or functionally identical elements.

### DETAILED DESCRIPTION

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically”, “typically”, “more typically”, “preferably”, “more preferably” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

As used herein, the term “biomass” refers to the mass of biological organisms comprised in the solution. Typically, said biological organisms in accordance with the present invention are one or more types of prokaryotic or eukaryotic organisms and, preferably bacteria or yeast. More preferably, the said biomass comprises bacteria, even more preferably genetically modified Escherichia coli, which are cultivated in a cultivation medium, preferably a cultivation medium comprising at least one carbon source, at least one nitrogen source and inorganic nutrients. In a further embodiment, the methods of the invention are applied to separate oligosaccharides, disaccharides and monosaccharides produced from macromolecular biomass, such as wood, straw, stalks and other plant material containing lignin, cellulose and/or starch, or from macromolecular biomass or animal or microbial origin, such as chitin containing substances, polysaccharides and the like from the remainders of said macromolecular biomass.

The easiest way to assess the success of separating the biomass and the oligosaccharide(s), disaccharide(s) and/or monosaccharide(s) is to monitor that the permeate of the first membrane filtration is optically clear. Unsuccessful separation will result in biomass being detected in the optical check of the permeate, and the presence of adsorbing agent like black active carbon in the permeate will also easily be detected in the optical check and indicate a leak or failure of the membrane filtration equipment.

As used herein, the term “oligosaccharide” refers to a saccharide polymer containing a small number of typically three to ten of monosaccharides (simple sugars). Preferably, said oligosaccharide comprises human milk oligosaccharide, preferably neutral, acidic nonfucosylated and/or acidic fucosylated, more preferably 2′-fucosyllactose, Difucosyllactose, Lacto-N-tetraose, Lacto-N-neotetraose, LNFP I, LNFP II, LNFP III, LNFP V, LNDFH I, LNDFH II and/or sialic acid containing human milk oligosaccharides such as but not limited to 3′-sialyllactose and/or 6′-sialyllactose, even more preferably 2′-fucosyllactose.

As used herein, the term “disaccharide” refers to a saccharide consisting of two monosaccharides, for example lactose that consists of a glucose and a galactose moiety, or saccharose that is made from one glucose and one fructose molecule.

As used herein, the term “monosaccharide” refers to a simple sugar, preferably a sugar molecule comprising 5 or 6 carbon atoms, for example glucose, fructose, galactose or fucose.

The term “adsorbing agent” as used herein refers to an element configured to provide the adhesion of atoms, ions or molecules from a gas, liquid or dissolved solid to a surface. The term “adhesion” refers to the tendency of dissimilar particles or surfaces to cling to one another. Preferably, the adsorbing agent is configured to provide adhesion for color components. Preferably, the adsorbing is active carbon.

As used herein, the term “microfiltration” refers to a type of physical filtration process where a fluid comprising undesired particles, for example contaminated fluid is passed through a special pore-sized membrane to separate microorganisms and suspended particles from process liquid, particularly larger bacteria, yeast, and any solid particles. Microfiltration membranes haves a pore size of 0.1 μm to 10 μm. Thereby, such membranes have a cut-off for a molecular mass of more than 100000 kDa.

As used herein, the term “ultrafiltration” refers to a type of physical filtration process where a fluid comprising undesired particles, for example contaminated fluid is passed through a special pore-sized membrane to separate microorganisms and suspended particles from process liquid, particularly bacteria, macromolecules, proteins, larger viruses. Ultrafiltration membranes have typically a pore size of 2 nm to 100 nm and have a cut-off for a molecular mass of 2 kDa to 250000 kDa. The principles underlying ultrafiltration are not fundamentally different from those underlying microfiltration. Both of these methods separate based on size exclusion or particle retention, but differ in their separation ability depending on the size of the particles.

According to the present inventive methods, first membrane filtration is carried out preferably by means of a polymeric microfiltration membrane or polymeric ultrafiltration membrane having a cut-off equal to or above 4 kDa, preferably in the range of 10 kDa to 200 nm, more preferably in the range of 50 kDa to 200 nm and even more preferably in the range of 50 kDa to 100 nm. Further, said second membrane filtration is preferably carried out by means of an ultrafiltration membrane having a cut-off in the range of 1 kDa to 10 kDa, preferably in the range of 2 kDa to 10 kDa and more preferably in the range of 4 kDa to 5 kDa.

The cut-off of a filtration membrane typically refers to retention of 90% of a solute of a given size or molecular mass, e.g. 90% of a globular protein with x kDa are retained by a membrane with a cut-off of x kDa. These cut-off values can be measured for example by the use of defined dextranes or polyethylene glycols and analyzing the retentate, the permeate and the original solution also called feed with a GPC gel permeation chromatography analysator using methods and parameters common in the art.

As used herein, the term “cross-flow filtration” refers to a type of filtration where the majority of the feed flow travels tangentially across the surface of the filter, rather than into the filter, at positive pressure relative to the permeate side. The principal advantage of this is that the filter cake which can blind the filters in other methods is not building up during the filtration process, increasing the length of time that a filter unit can be operational. It can be a continuous process, unlike batch-wise dead-end filtration. For large scale applications, a continuous process is preferable. This type of filtration is typically selected for feeds containing a high proportion of small particle size solids where the permeate is of most value because solid material can quickly block (blind) the filter surface with dead-end filtration. According to the present disclosure, said cross-flow microfiltration or cross-flow ultrafiltration includes a cross-flow speed in the range of 0.5 m/s to 6.0 m/s, preferably in the range of 2.0 m/s to 5.5 m/s and more preferably in the range of 3.0 m/s to 4.5 m/s. In case of a membrane made of ceramics, the cross-flow speed may be higher than in case of a membrane made of a polymeric material depending on the respective geometry of the membrane. For example, in case of a flat polymeric membrane such as a polymeric membranes in flat sheet modules, the cross-flow speed is 0.5 m/s to 2.0 m/s and preferably 1.0 m/s to 1.7 m/s. and more preferably 1.0 to 1.5 m/s. Depending on the particular set-up and the particular solution comprising the biomass even cross-flow speeds of 1.0 m/s or less may be used in some cases, yet the filtration may turn into a dead end filtration when the cross-flow speeds are too low.

The term “cut-off” as used herein refers to the exclusion limit of a membrane which is usually specified in the form of MWCO, molecular weight cut off, with units in Dalton. It is defined as the minimum molecular weight of a solute, for example a globular protein that is retained to 90% by the membrane. The cut-off, depending on the method, can be converted to so-called D90, which is then expressed in a metric unit.

In a first step (FIG. 1, step S10), a solution comprising biomass and at least one oligosaccharide is provided. Said at least one oligosaccharide comprises human milk oligosaccharide, preferably 2′-fucosyllactose. Preferably, said solution comprising biomass and oligosaccharide is obtained by cultivation of one or more types of cells in a cultivation medium. Thus, said solution may also be called fermentation broth in a preferred embodiment. The cultivation medium is preferably a cultivation medium comprising at least one carbon source, at least one nitrogen source and inorganic nutrients. More preferably, the fermentation broth or solution comprising biomass and the at least one oligosaccharide is obtained by microbial fermentation, preferably aerobic microbial fermentation. A microorganism capable of producing the oligosaccharide may be a yeast or a bacterium, for example from the group consisting of the genera Escherichia, Klebsiella, Helicobacter, Bacillus, Lactobacillus, Streptococcus, Lactococcus, Pichia, Saccharomyces and Kluyveromyces or as described in the international patent application published as WO 2015/032412 or the European patent application published as EP 2 379 708, preferably a genetically modified E. coli strain, more preferably a genetically modified E. coli strain that is deficient in the lacZ gene (lacZ-) and suitable for the production of substances for human nutrition, that is cultivated in an aqueous nutrient medium under controlled conditions, favorable for biosynthesis of the oligosaccharide, for example as disclosed in EP 2 379 708, EP 2 896 628 or U.S. Pat. No. 9,944,965. The aqueous nutrient medium comprises at least one carbon source (e.g. glycerol or glucose) which is used by the microorganism for growth and/or for biosynthesis of the oligosaccharide. In addition, the nutrient medium also contains at least one nitrogen source, preferably in the form of an ammonium salt, e.g. ammonium sulfate, ammonium phosphate, ammonium citrate, ammonium hydroxide etc., which is necessary for the growth of the microorganisms. Other nutrients in the medium include e.g. one or several phosphate salts as phosphor source, sulfate salts as sulfur source, as well as other inorganic or organic salts providing e.g. Mg, Fe and other micronutrients to the microorganisms. In many cases, one or more vitamins, e.g. thiamin, has to be supplemented to the nutrient medium for optimum performance. The nutrient medium may optionally contain complex mixtures such as yeast extract or peptones. Such mixtures usually contain nitrogen-rich compounds such as amino acids as well as vitamins and some micronutrients.

The nutrients can be added to the medium at the beginning of the cultivation, and/or they can also be fed during the course of the process. Most often the carbon source(s) are added to the medium up to a defined, low concentration at the beginning of the cultivation. The carbon source(s) are then fed continuously or intermittently in order to control the growth rate and, hence, the oxygen demand of the microorganisms. Additional nitrogen source is usually obtained by the pH control with ammonia (see below). It is also possible to add other nutrients mentioned above during the course of the cultivation.

In some cases, a precursor compound is added to the medium, which is necessary for the biosynthesis of the oligosaccharide. For instance, in the case of 2′-Fucosyllactose, lactose is usually added as a precursor compound. The precursor compound may be added to the medium at the beginning of the cultivation, or it may be fed continuously or intermittently during the cultivation, or it may be added by a combination of initial addition and feeding.

The cells are cultivated under conditions that enable growth and biosynthesis of the oligosaccharide in a stirred tank bioreactor. A good oxygen supply in the range of 50 mmol O₂/(l*h) to 180 mmol O₂/(l*h) to the microbial cells is essential for growth and biosynthesis, hence the cultivation medium is aerated and vigorously agitated in order to achieve a high rate of oxygen transfer into the liquid medium. Optionally, the air stream into the cultivation medium may be enriched by a stream of pure oxygen gas in order to increase the rate of oxygen transfer to the cells in the medium. The cultivation is carried out at 24° C. to 41° C., preferably 32° C. to 39° C., the pH value is set at 6.2 to 7.2, preferably by automatic addition of NH₃ (gaseous or as an aqueous solution of NH₄OH).

In some cases, the biosynthesis of the oligosaccharide needs to be induced by addition of a chemical compound, e.g. Isopropyl β-D-1-thiogalactopyranoside (IPTG) for example as in the European patent application published as EP 2 379 708. The inducer compound may be added to the medium at the beginning of the cultivation, or it may be fed continuously or intermittently during the cultivation, or it may be added by a combination of initial addition and feeding.

Subsequently, the method of the invention proceeds to the adjustment of the pH value in a second step (FIG. 1, step S12). In said step, typically the pH value of the solution below 7 is lowered by adding at least one acid to the solution comprising biomass and the at least one oligosaccharide. The pH value of the solution is lowered to a target pH value preferably in the range of 3.0 to 5.5, more preferably in the range of 3.5 to 5 and even more preferably in the range of 4.0 to 4.5, such as 4.0 or 4.1. Said at least one acid is an acid selected from the group consisting of H₂SO₄, H₃PO₄, HCl, HNO₃ (preferably not in concentrated form) and CH₃CO₂H, or any other acid considered safe in production of food or feed; preferably the acid is selected from the group consisting of H₂SO₄, H₃PO₄, HCl and CH₃CO₂H. A mix of these acids may be used in one embodiment instead of a single of these acids.

Further, in another embodiment of the method of the invention, if the solution comprising biomass and the at least one oligosaccharide, at least one disaccharide or at least one monosaccharide already has a pH value below 7, preferably below pH 5.5, more preferably equal to or below pH 5.0 and even more preferably equal to or below pH 4.5, there will be no addition of any of these acids, and step S12 may be skipped and the methods of the invention for such solutions continues with Step S14.

The method then proceeds to the next step (FIG. 1, S14). In said step, one or more adsorbing agent is added to the solution comprising biomass and the at least one oligosaccharide. Preferably, the adsorbing agent is active carbon. Said adsorbing agent, preferably active carbon, is added in an amount in the range of 0.5% to 3% by weight, preferably in the range of 0.6% to 2.5% by weight and more preferably in the range of 0.7% to 2.0% by weight, such as 1.5%. In this respect, it has to be noted that the smaller the particles of the adsorbing agent are, the better the adsorption characteristics are. Said adsorbing agent, preferably active carbon, is added as a powder having a particle size distribution with a diameter d50 in the range of 2 μm to 25 μm, preferably in the range of 3 μm to 20 μm and more preferably in the range of 3 μm to 7 μm such as 5 μm. More preferably, said adsorbing agent, preferably active carbon, is added as a suspension of the powder in water. Preferably, adding said adsorbing agent, preferably active carbon, to the solution is carried out after adding the at least one acid to the solution. Alternatively, adding said adsorbing agent, preferably active carbon, to the solution may be carried out before adding the at least one acid to the solution. With other words, the order of steps S12 and S14 may be changed and the order thereof is not fixed. Yet if the order is first setting of the pH below 7 to the desired pH value and then adding one or more adsorbing agents, preferably active carbon, will generate the best results with respect to protein removal and decolorization. In a preferred embodiment addition of the at least one acid antedates the addition of the at least one adsorbing agent, preferably active carbon.

In a preferred embodiment of the methods of the invention, the steps S12 and S14 are both performed and in the order S12 followed by S14.

The method then proceeds with first membrane filtration, preferably a micro- or ultrafiltration in a further step (FIG. 1, step S16) including a time suitable for the adhesion of color components to the one or more adsorbing agents before the separation. The first membrane filtration is carried out so as to separate the biomass and the one or more adsorbing agents from the solution comprising the at least one oligosaccharide, at least one disaccharide and/or at least one monosaccharide, and by this removing the biomass and also reducing the color components and protein in the resulting solution also called permeate comprising the oligosaccharides, disaccharides and/or monosaccharides. Basically, step S16 includes microfiltration or ultrafiltration. However, as there is a smooth transition between microfiltration and ultrafiltration and both can be used by the skilled artisan to the purpose of separating biomass, adsorbing agent and protein on one side and the permeate containing the bulk of the desired one or more oligosaccharides, one or more disaccharides and/or one or more monosaccharides on the other side. The filtration in step S16 may also be an ultrafiltration as an alternative to microfiltration. Said microfiltration or ultrafiltration is preferably carried out as cross-flow microfiltration or cross-flow ultrafiltration to improve membrane performance and reduce membrane abrasion. The details of the filtration in step S16 will be explained below. Said cross-flow microfiltration or cross-flow ultrafiltration includes a cross-flow speed in the range of 0.5 m/s to 6.0 m/s, preferably in the range of 2.0 m/s to 5.5 m/s and more preferably in the range of 3.0 m/s to 4.5 m/s, such as 4.0 m/s. In one embodiment the cross-flow speed is equal to or below 3.0 m/s, preferably between and including 1.0 and 2.0. One advantageous of the inventive method, use and the apparatus of the invention is that lower cross-flow speeds can be used to achieve good separation preferably of protein components of the solution from any oligosaccharides, disaccharides or monosaccharides. Thus, energy and equipment cost can be reduced, wear and tear on equipment and abrasion of the filtration membrane are also reduced. Said first membrane filtration, preferably microfiltration or ultrafiltration, is carried out at a temperature of the solution in the range of 8° C. to 55° C., preferably in the range of 10° C. to 50° C. and more preferably in the range of 30° C. to 40° C., such as 38° C. Said microfiltration or ultrafiltration is carried out by means of a ceramic or polymeric microfiltration membrane or ceramic ultrafiltration membrane having a pore size in the range of 20 nm to 800 nm, preferably in the range of 40 nm to 500 nm and more preferably in the range of 50 nm to 200 nm, such as 100 nm. Said ceramic material is or has at least one layer of at least one ceramic material selected from the group consisting of: Titanium dioxide (TiO₂), Zirconium dioxide (ZrO₂), Silicon carbide (SiC) and Aluminium oxide (Al₂O₃). Alternatively, said microfiltration or ultrafiltration is carried out by means of a polymeric microfiltration membrane or polymeric ultrafiltration membrane having a cut-off in the range of 10 kDa to 200 nm, preferably in the range of 50 kDa to 200 nm and more preferably in the range of 50 kDa to 100 nm. Said polymeric material is at least one polymeric material selected from the group consisting of: polyethersulfone, polysulfone, polypropylene, polyvinylidene fluoride, polyacrylonitrile, polyvinylidene fluoride. Said first membrane filtration, preferably microfiltration or ultrafiltration, is carried out after a predetermined time after the adsorbing agent, preferably active carbon, has been added to the solution. Thus, ensures adhesion of color components. Typically, the time needed for mixing of the solution with the added adsorbing agent until a homogenous distribution of the adsorbing agent, preferably active carbon, in the solution has been reached may suffice to allow for the adhesion of the color components, yet a longer incubation time can be used to maximize this. In one embodiment, said predetermined time is at least 2 min, preferably at least 10 min and more preferably at least 20 min such as 25 min or 30 min.

In one embodiment, the method of the invention typically then proceeds with a second membrane filtration step (FIG. 1, step S18). Preferably an ultrafiltration of the solution comprising oligosaccharides, disaccharides and/monosaccharides obtained by the first membrane filtration of step S16 is carried out. In other words, an ultrafiltration of the permeate derived from the first membrane filtration in step S16 is carried out. Preferably, said second membrane filtration, preferably ultrafiltration, is carried out by means of an ultrafiltration membrane having a cut-off in the range of 1.5 kDa to 10 kDa, preferably in the range of 2 kDa to 10 kDa and more preferably in the range of 4 kDa to 5 kDa. In a particularly preferred embodiment, membranes with a cut-off of 4 kDa or 5 kDa are suitable. Said ultrafiltration membrane is at least partially made of a polymeric material. Said polymeric material is at least one polymeric material selected from the group consisting of: polyethersulfone, polyacrylonitrile, cellulose acetate. Said second membrane filtration, preferably ultrafiltration, is carried out at a temperature of the solution being in the range of 5° C. to 15° C., preferably in the range 8° C. to 13° C. and more preferably in the range 8° C. to 12° C., such as 10° C.

FIG. 2 displays the sequence of steps of the inventive methods with the time suitable for the adhesion of color components to the one or more adsorbing agents before the separation shown as a separate step (S15 in FIG. 2). Such a separate incubation step may be favorable when long times for sufficient adhesion of the undesired compounds to the adsorbing agent are required. Further, FIG. 2 depicts for the first membrane filtration (which was S16 in FIG. 1) as a step with three parts; the three steps of first membrane filtration being first diafiltration, concentrating and then optionally a second diafiltration. These are shown as S16-1, S16-2 and S16-3, respectively, in FIG. 2. The other steps are as in FIG. 1.

In another embodiment the steps S10 to S18 are performed wherein instead of an at least one oligosaccharide, at least one monosaccharide, at least one disaccharide or a mixture of at least one monosaccharide, at least one disaccharide and/or at least one oligosaccharide are present in place of the at least one oligosaccharide.

For the avoidance of doubt, any reference to the protein content of the solution or the permeate or retentate is referring to free protein in the solution/permeate/retentate, i.e. the protein found extracellularly and not the protein contained in the biomass if any. During fermentation and also subsequent handling and membrane filtrations, protein may be liberated from biomass and then be considered free protein.

For the avoidance of doubt, any reference to the at least one oligosaccharide, at least one disaccharide and/or at least one monosaccharide the solution or the permeate or retentate is referring to free the at least one oligosaccharide, at least one disaccharide and/or at least one monosaccharide in the solution/permeate/retentate, i.e. the at least one oligosaccharide, at least one disaccharide and/or at least one monosaccharide found extracellularly and not the ones contained in the biomass if any. During fermentation and also subsequent handling and membrane filtrations, the at least one oligosaccharide, at least one disaccharide and/or at least one monosaccharide may be liberated from biomass and then be considered free the at least one oligosaccharide, at least one disaccharide and/or at least one monosaccharide in the solution.

In a preferred embodiment, the step of carrying out first membrane filtration, preferably a microfiltration or ultrafiltration, so as to separate the biomass from the solution comprising the at least one oligosaccharide, at least one disaccharide and/or at least one monosaccharide is to be understood as a step of separating the biomass from the at least one oligosaccharide, at least one disaccharide and/or at least one monosaccharide, wherein the majority of the at least one oligosaccharide, at least one disaccharide and/or at least one monosaccharide is found in the permeate of the first membrane filtration following the separation of biomass.

In a preferred embodiment, the first membrane filtration is followed by an ultrafiltration, then optionally followed by a nanofiltration, ion exchange and/or reverse osmosis.

Summarizing, the present invention includes the following embodiments, wherein these include the specific combinations of embodiments as indicated by the respective interdependencies defined therein.

### Further Embodiments

Embodiment 1: A method for separating biomass from a solution comprising biomass and at least one oligosaccharide, at least one disaccharide and/or at least one monosaccharide, comprising the steps of:
a. providing the solution comprising biomass and at least one oligosaccharide, at least one disaccharide and/or at least one monosaccharide,
b. if the pH value is equal to or above pH 7.0, lowering the pH value of the solution below 7.0 by adding at least one acid to the solution comprising biomass and the at least one oligosaccharide, at least one disaccharide and/or at least one monosaccharide,
c. adding one or more adsorbing agents to the solution comprising biomass and at least one oligosaccharide, at least one disaccharide and/or at least one monosaccharide,
d. Optionally an incubation step sufficient for the one or more adsorbing agents to bind the color components in the solution, and
e. carrying out first membrane filtration, preferably a microfiltration or ultrafiltration, to the effect that the biomass and the one or more adsorbing agents are separated from the solution comprising the at least one oligosaccharide, at least one disaccharide and/or at least one monosaccharide.

Embodiment 1A: A method for separating biomass from a solution comprising biomass and at least one oligosaccharide, at least one disaccharide and/or at least one monosaccharide, comprising the steps of:
a. providing the solution comprising biomass and at least one oligosaccharide, at least one disaccharide and/or at least one monosaccharide,
b. optionally adding one or more adsorbing agents to the solution
c. setting the pH value of the solution below 7.0, preferably below 5.5, more preferably below 5.0 and even more preferably to pH 4.5 or below, by adding at least one acid to the solution comprising biomass and the at least one oligosaccharide, at least one disaccharide and/or at least one monosaccharide,
d. adding one or more adsorbing agents to the solution comprising biomass and at least one oligosaccharide, at least one disaccharide and/or at least one monosaccharide in an amount suitable to remove color components and the majority of the extracellular protein in the solution,
e. Optionally an incubation step sufficient for the one or more adsorbing agents to bind the color components in the solution, and
f. carrying out first membrane filtration, preferably a microfiltration or ultrafiltration, to the effect that the biomass is separated from the solution comprising the majority of at least one oligosaccharide, at least one disaccharide and/or at least one monosaccharide.

Embodiment 1B: A method for separating biomass from a solution comprising biomass and at least one oligosaccharide, at least one disaccharide and/or at least one monosaccharide, comprising the steps of:
a. providing the solution comprising biomass and at least one oligosaccharide, at least one disaccharide and/or at least one monosaccharide,
b. lowering the pH value of the solution below 7.0 by adding at least one acid to the solution comprising biomass and the at least one oligosaccharide, at least one disaccharide and/or at least one monosaccharide,
c. adding one or more adsorbing agents to the solution comprising biomass and at least oligosaccharide, at least one disaccharide and/or at least one monosaccharide,
d. Optionally an incubation step sufficient for the one or more adsorbing agents to bind the color components in the solution, and
e. carrying out first membrane filtration, preferably a microfiltration or ultrafiltration, so as to separate the biomass from the solution comprising the at least one oligosaccharide, at least one disaccharide and/or at least one monosaccharide.

Embodiment A1: An apparatus comprising
i. a solution containing biomass, at least one adsorbing agent, preferably active carbon and at least one oligosaccharide and/or at least one disaccharide and/or at least one monosaccharide, wherein the pH value of the solution is below 7,
ii. a first filtration membrane, preferably a microfiltration or an ultrafiltration membrane,
iii. means to carry out a first membrane filtration across said first filtration membrane, preferably a microfiltration or ultrafiltration, to generate a permeate containing the bulk of the oligosaccharides, disaccharides and or monosaccharides, and
iv. means to separate the permeate of the first membrane filtration from the solution as described in i. above,
   and further optionally comprising:
v. means to transport said permeate of the first membrane filtration to a second filtration membrane,
vi. means to adjust the temperature of the permeate to a temperature below 20° C.,
vii. a second filtration membrane, preferably an ultrafiltration membrane,
viii. means to carry out a second membrane filtration, preferably a ultrafiltration, at a temperature below 20° C., and
ix. means to keep separate the permeate of the second membrane filtration from the permeate of the first membrane filtration,
   wherein the surfaces of the parts of the apparatus that are in contact with the solution or any of the permeates are made of material suitable for the production of food and are tolerant to pH values as low as pH 3.5.

Embodiment A2: An apparatus comprising
i. a vessel holding a solution containing biomass and at least one oligosaccharide and/or at least one disaccharide and/or at least one monosaccharide
ii. means to adjust the temperature of said solution to a temperature between 5° C. and 70° C.;
iii. a measuring system to measure the pH value of the solution in the vessel;
iv. means to set the pH value of the solution to a value below 7.0, preferably a target pH value lower than 5.5, wherein preferably the means to set the pH are suitable for the addition of at last one acid,
v. Means to add at least one adsorbing agent, preferably active carbon, to the solution,
vi. Means to generate an essentially homogenous distribution of the adsorbing agent in the solution
vii. a first filtration membrane, preferably a microfiltration or an ultrafiltration membrane,
viii. means to carry out with the help of said first filtration membrane a first membrane filtration, preferably a microfiltration or ultrafiltration, of the solution with a pH value below 7.0 and containing biomass, at least one adsorbing agent, preferably active carbon and at least one oligosaccharide and/or at least one disaccharide and/or at least one monosaccharide, and wherein the means are suitable to generate a permeate containing the bulk of the oligosaccharides, disaccharides and/or monosaccharides, and
ix. means to collect, transport and optionally store the permeate of the first membrane filtration from the solution with a pH value below 7.0 and containing biomass, at least one adsorbing agent, preferably active carbon and at least one oligosaccharide and/or at least one disaccharide and/or at least one monosaccharide,
   and further optionally comprising:
x. means to transport said permeate of the first membrane filtration to a second filtration membrane,
xi. means to adjust the temperature of the first permeate to a temperature below 20° C.,
xii. a second filtration membrane, preferably an ultrafiltration membrane,
xiii. means to carry out a second membrane filtration, preferably a ultrafiltration, at a temperature below 20° C., and
xiv. means to separate the permeate of the second membrane filtration from the permeate of the first membrane filtration,

wherein the surfaces of the parts of the apparatus that are in contact with the solution or any of the permeates are made of material suitable for the production of food and are tolerant to pH values as low as pH 3.5.

Embodiment B1

Method for reducing wear and tear on and/or energy consumption of membrane filtration equipment used in the separation of biomass from a solution comprising at least one oligosaccharide, at least one disaccharide and/or at least one monosaccharide, wherein the method comprises these steps:
a. providing the solution comprising biomass and saccharides,
b. if the pH value is pH 7.0 or higher lowering the pH value of the solution below 7 by adding at least one acid to the solution comprising biomass and comprising at least one oligosaccharide, at least one disaccharide and/or at least one monosaccharide,
c. adding one or more adsorbing agents to the solution comprising biomass and oligosaccharide,
d. Optionally as required an incubation step sufficient for the one or more adsorbing agents to bind the color components in the solution, and
e. carrying out first membrane filtration so as to separate the biomass from the solution comprising the comprising at least one oligosaccharide, at least one disaccharide and/or at least one monosaccharide at cross-flow speeds of no more than 3 m/s.

Embodiment 2: The method according to any of the embodiments 1, 1A, 1B or B1, or the apparatus according to embodiment A1 or A2, wherein the adsorbing agent is active carbon.

Embodiment 3: The method or apparatus according to any of the previous embodiments, wherein the pH value of the solution is lowered to a pH value in the range of 3.0 to 5.5, preferably the range of 3.5 to 5 and more preferably the range of 4.0 to 4.5.

Embodiment 4: The method or apparatus according to any of the previous embodiments, wherein said at least one acid is an acid selected from the group consisting of H₂SO₄, H₃PO₄, HCl, HNO₃ and CH₃CO₂H.

Embodiment 5: The method or apparatus according to any of the previous embodiments, wherein said adsorbing agent, preferably active carbon, is added in an amount in the range of 0.5% to 3% by weight, preferably in the range of 0.75% to 2.5% by weight and more preferably in the range of 1.0% to 2.0% by weight.

Embodiment 6: The method or apparatus according to any of the previous embodiments, wherein said adsorbing agent, preferably active carbon, is added as a powder having a particle size distribution with a diameter d50 in the range of 2 μm to 25 μm, preferably in the range of 3 μm to 20 μm and more preferably in the range of 3 μm to 7 μm.

Embodiment 7: The method or apparatus according embodiment 6, wherein said adsorbing agent, preferably active carbon, is added as a suspension of the adsorbing agent powder in water.

Embodiment 8: The method or apparatus according to any of the previous embodiments, wherein adding said adsorbing agent, preferably active carbon, to the solution is carried out when the pH value of the solution is below 7, and while at least one acid continues to be added to the solution or after adding the at least one acid to the solution has been completed.

Embodiment 9: The method or apparatus according to any of the previous embodiments except embodiment 8, wherein adding said adsorbing agent, preferably active carbon, to the solution is carried out before adding the at least one acid to the solution.

Embodiment 10: The method or apparatus according to any of the previous embodiments, wherein said solution comprising biomass and one or more oligosaccharides, one or more disaccharides and/or one or more monosaccharides is obtained by cultivation of one or more types of cells, preferably bacteria or yeast, more preferably bacteria, even more preferably genetically modified Escherichia coli, in a cultivation medium, preferably a cultivation medium comprising at least one carbon source, at least one nitrogen source and inorganic nutrients.

Embodiment 11: The method or apparatus according to any of the previous embodiments, wherein providing the solution comprising biomass and at least one oligosaccharide, one or more disaccharides and/or one or more monosaccharides includes preparing said solution by means of microbial fermentation.

Embodiment 12: The method or apparatus according to any of the previous embodiments except embodiment B1, wherein said first membrane filtration is carried out as cross-flow microfiltration or cross-flow ultrafiltration.

Embodiment 13: The method or apparatus according to embodiment 12, wherein said cross-flow microfiltration or cross-flow ultrafiltration includes a cross-flow speed in the range of 0.5 m/s to 6.0 m/s, preferably in the range of 2.0 m/s to 5.5 m/s and more preferably in the range of 2.2 m/s to 4.5 m/s and even more preferably in the range of 2.5 to 4.5.

Embodiment 14: The method or apparatus according to any of the previous embodiments, wherein said first membrane filtration is carried out at a temperature of the solution in the range of 8° C. to 55° C., preferably in the range of 10° C. to 50° C. and more preferably in the range of 30° C. to 40° C.

Embodiment 15: The method or apparatus according to any of the previous embodiments, wherein said first membrane filtration is carried out by means of a ceramic microfiltration membrane or ceramic ultrafiltration membrane having a pore size in the range of 20 nm to 800 nm, preferably in the range of 40 nm to 500 nm and more preferably in the range of 50 nm to 200 nm.

Embodiment 16: The method or apparatus according to embodiment 15, wherein said ceramic material is at least one ceramic material selected from the group consisting of: TiO₂, ZrO₂, SiC and Al₂O₃.

Embodiment 17: The method or apparatus according to any of the previous embodiments, wherein said first membrane filtration is carried out by means of a polymeric microfiltration membrane or polymeric ultrafiltration membrane having a cut-off in the range of 10 kDa to 200 nm, preferably in the range of 50 kDa to 200 nm and more preferably in the range of 50 kDa to 100 nm.

Embodiment 18: The method or apparatus according to embodiment 17, wherein said polymeric material is at least one polymeric material selected from the group consisting of: polyethersulfone, polysulfone, polypropylene, polyvinylidene fluoride, polyacrylonitrile, polyvinylidene fluoride.

Embodiment 19: The method or apparatus according to any of the previous embodiments, wherein said first membrane filtration is carried out after a predetermined time after the adsorbing agent, preferably active carbon, has been added to the solution.

Embodiment 20: The method or apparatus according to embodiment 19, wherein said predetermined time is at least 2 min, preferably at least 10 min and more preferably at least 20 min.

Embodiment 21: The method of any of the previous embodiments, wherein the first membrane filtration comprises preferably two, more preferably three steps: a first diafiltration step, a concentrating step and optionally a second diafiltration step, each as disclosed in detail in this application.

Embodiment 22: The method according to any one of the previous embodiments, further comprising carrying out a second membrane filtration, of the solution comprising at least one oligosaccharide, one or more disaccharides and/or one or more monosaccharides obtained by the first membrane filtration, preferably an ultrafiltration of the permeate of the first membrane filtration.

Embodiment 23: The method according to embodiment 22, wherein said second membrane filtration is an ultrafiltration and is carried out by means of an ultrafiltration membrane having a cut-off in the range of 1.0 kDa to 10 kDa, preferably in the range of 2 kDa to 10 kDa and more preferably in the range of 4 kDa to 5 kDa.

Embodiment 24: The method according to embodiment 23, wherein said ultrafiltration membrane is at least partially made of a polymeric material.

Embodiment 25: The method according to embodiment 24, wherein said polymeric material is at least one polymeric material selected from the group consisting of: polyethersulfone, polyacrylonitrile, cellulose acetate.

Embodiment 26: The method according to any one of embodiments 22 to 25, wherein said second membrane filtration, preferably ultrafiltration, is carried out at a temperature of the solution being in the range of 5° C. to 15° C., preferably in the range 8° C. to 13° C. and more preferably in the range 8° C. to 12° C.

Embodiment 27: The method according to any one of embodiments 22 to 26, wherein the solution comprising oligosaccharide obtained by the first membrane filtration is brought to a temperature of below 20° C. before and preferably maintained a temperature of below 20° C. during said second membrane filtration.

Embodiment 27: The method or apparatus according to any one of the previous embodiments, wherein said at least one oligosaccharide comprises human milk oligosaccharide, preferably 2′-fucosyllactose, 6′-sialyllactose or Lacto-N-tetraose, and more preferably 2′-fucosyllactose.

Embodiment 28:

Any of the previous embodiments wherein biomass is macromolecular biomass.

Embodiment 29:

Embodiment 28 wherein macromolecular biomass comprises
wood, straw, stalks and other plant material containing lignin, lignocellulose, cellulose and/or starch; and/or
macromolecular biomass of animal and/or microbial origin, preferably chitin containing substances and/or polysaccharides.

Embodiment 30:

Any of the previous embodiment wherein the solution comprising biomass and at least one oligosaccharide, at least one disaccharide and/or at least one monosaccharide comprises a mixture of at least two of the following:
at least one oligosaccharide, and
at least one disaccharide.

### FIGURES

FIG. 1 shows a block diagram of a method for separating biomass from a solution comprising biomass and at least one oligosaccharide according to the present invention.

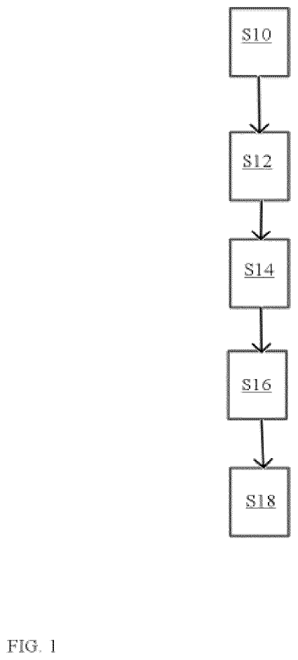

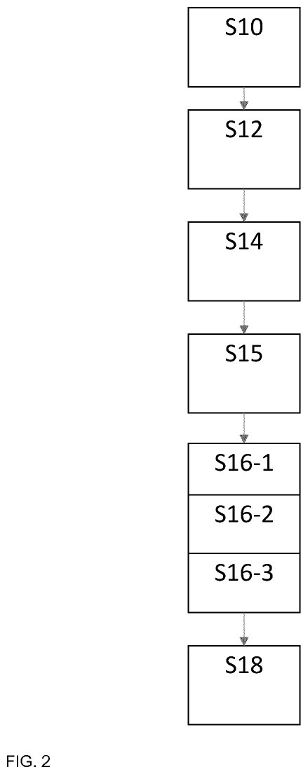

### EXAMPLES

The method according to the present invention will be described in further detail below. Whatsoever, the Examples shall not be construed as limiting the scope of the invention.

### Example 1

A fermentation broth as a complex solution comprising biomass and at least one oligosaccharide has been prepared by standard methods in the amount of 2.4 kg. The pH value thereof has been lowered to 4±0.1 by means of adding 92 g 10% sulfuric acid. Thereafter, 98 g of a 30% suspension of active carbon Carbopal Gn-P (Donau Carbon GmbH, Gwinnerstraβe 27-33, 60388 Frankfurt am Main, Germany), which is food safe, has been added and stirred for 20 min.

The thus prepared solution has been supplied to the process apparatus, a semi-automatic MF lab unit from Sartorius AG, Otto-Brenner-Str. 20, 37079 Goettingen, Germany, modified for the purpose, and heated to 37° C. in a circulating manner with closed permeate. For separation purposes, the process apparatus included a mono channel element (from Atech Innovations GmbH, Gladbeck, Germany) having an outer diameter of 10 mm, an inner diameter of 6 mm, a length of 1.2 m and a membrane made of Al₂O₃ having a pore size of 50 nm. As soon as the circulation of the solution is running and the solution comprises the target temperature of 37° C., the discharging of the permeate has been started and the control of the trans membrane pressure has been activated.

After terminating of the inventive method, the process apparatus has been stopped, the concentrate has been disposed and the process apparatus has been cleaned. Cleaning has been carried out by means of 0.5% to 1% NaOH at a temperature of 50° C. to 80° C., wherein the NaOH has been subsequently removed by purging.

In a preferred embodiment, the first membrane filtration of the inventive methods includes three steps as will be explained in further detail below. The first step includes a first diafiltration having a factor of 0.5 (amount of diafiltration water=starting amount of fermentation broth×diafiltration factor). During diafiltration, the amount of water added is identical to the amount of permeate discharged. The first step is a continuing step and the volume in the feed vessel is thus kept constant. The second step includes concentrating of the fermentation broth with the factor 2 by stopping the feed of diafiltration water and the level will decrease down to the target value (target value=volume or mass at the beginning of the fermentation broth/concentrating factor).

Subsequently, the third step includes a second diafiltration. The permeates collected during these three steps are typically combined to form the permeate referred to in the tables below. By means of these three steps a lower dilution of the product within the permeate and an increased yield of ≥95% are realized. By increasing the factor of the second diafiltration, the yield may even be increased.

The following analytical methods have been carried out.
HPLC for the determination of the product, i.e. human milk oligosaccharides, and secondary components
Drying balances for measuring the dry content
APHA for measuring the color using standard methods, for example DIN EN ISO 6271
Bradford protein assay for measuring the concentration of protein.

Some experiments have been made with different fermentation broths as these may not be stored over a longer period of time. In order to be able to determine whether the method correctly works and provides the announced advantages, experiments have been made:
without adjustment of pH value and without adding active carbon,
without adjustment of pH value and with adding active carbon,
after adjustment of pH value and without adding active carbon,
after adjustment of pH value and with adding active carbon,
after adding active carbon and then adjustment of pH value.

Hereinafter, the following abbreviations are used:
AC=Active Carbon
UF=Ultrafiltration
DF=Diafiltration factor (ratio: amount of water and start volume)
CF=Concentration factor (ratio between start volume and final volume)
DP=Pressure drop along the module (p_{feed}−pᵣₑₜₑₙₜₐₜₑ)
Flux=Permeate flow rate per m² and hour (l/m² h)
Cross-flow velocity=linear speed of the suspension in membrane channels (m/s)
Membrane load=amount of permeate produced by 1 m² of membrane area (m³/m²)

Further, regarding the liquid separation, the following symbols and explanations are used.

| Symbol | Meaning | Unit | Definition |
|---|---|---|---|
| Letters | | | |
| CF | Concentration factor | — | m_{R,t=0}/m_{R} |
| DF | Diafiltration factor | — | m_{P}/m_{R,t=0} |
| J | Flux | LMH = L m⁻² h⁻¹ | |
| m | Mass | kg | |
| p | Pressure | bar | |
| R | Retention | — | 1 − Cₚₑᵣₘₑₐₜₑ/Cᵣₑₜₑₙₜₐₜₑ |
| TMP | Trans-membrane | bar | (p_{feed} + pᵣₑₜₑₙₜₐₜₑ)/ |
| | pressure | | 2 − pₚₑᵣₘₑₐₜₑ |

Still further, in the following tables, the term “Series” refers to the respective experimental number.

Table 1 shows the membrane performance depending on the pH value and active carbon. Different batches of fermentation broth originating from fermentations with varying parameters resulting in a solution with differing color components and different oligosaccharide and disaccharide compositions of the solution demonstrate the broad applicability of the methods of the invention.

TABLE 1

| | | | AC | | Flux | | | | cross |
|---|---|---|---|---|---|---|---|---|---|
| | | | | ads. | [kg/ | TMP | DP | Temp. | flow |
| Series | Batch | pH | [%] | [h] | m²h] | [bar] | [bar] | [°C.] | [m/s] |
| A1 | Batch 1 | 7.0 | | | 15 | 1.3 | 1.2 | 39.5 | 4.0 |
| | | 5.0 | | | 9 | 1.3 | 1.4 | 39.5 | 4.0 |
| | | 4.0 | | | 25 | 1.4 | 1.5 | 39.5 | 3.9 |
| | | 3.5 | | | 21 | 1.3 | 1.4 | 39.5 | 3.9 |
| A2 | Batch 2 | 7.0 | 0.0 | 0.0 | 12 | 1.3 | 1.1 | 39.4 | 4.0 |
| | | | 1.0 | 0.3 | 8 | 1.5 | 1.3 | 39.5 | 3.9 |
| | | 4.0 | 0.0 | 0.0 | 15 | 1.2 | 1.4 | 39.5 | 4.0 |
| | | | 1.0 | 0.3 | 77 | 1.2 | 1.3 | 41.0 | 3.7 |
| A3 | Batch 3 | 4.0 | 1.0 | 24 | 85 | 1.3 | 1.4 | 38.9 | 3.4 |
| | | | 2.0 | 24 | 75 | 1.6 | 1.7 | 38.8 | 3.8 |
| | | | 1.0 | 0.3 | 47 | 1.2 | 1.2 | 39.3 | 3.0 |
| | | | 2.0 | 0.3 | 37 | 1.3 | 1.3 | 39.4 | 3.0 |
| | | | 1.0 | 3 | 53 | 1.2 | 1.0 | 39.4 | 2.9 |

The abbreviation “ads. [h]” is the time after addition of the adsorbing agent to the solution before the start of the first membrane filtration in hours.

Series A 1 was done in the absence of any adsorbing agent yet at different pH values. Series A 2 was done at pH 7.0 and 4.0 and with or without active carbon. Series A3 was done at pH 4 and varying amounts of active carbon and differing cross-flow speeds as indicated. For these three series, only a first diafiltration step with DF=1 and a concentrating step with CF=2 were performed and then the first membrane filtration was stopped and the resulting solutions and remainder of the starting solutions analysed and results compared.

The following results are derivable from table 1:

The membrane performance has its maximum at a pH value of 4 at a cross-flow speed of 4 m/s. The membrane performance is reduced at a pH value of 7 with presence of 1% active carbon. whereas the membrane performance is enhanced at a pH value of 4 and with presence of 1% active carbon with a cross-flow speed of 4 m/s by a factor of approximately 4. An increase of the adsorption time after adding active carbon from 0.3 hours to 24 hours provides only a negligible enhancement of the membrane performance. An increase of the added amount of active carbon from 1% to 2% lowers the membrane performance. A reduction of the cross-flow speed from 4 m/s to 3 m/s reduces the membrane performance but the same is still higher than without presence of active carbon. A reduction of the cross-flow speed significantly reduces the electric power consumption and also reduces the risk of membrane abrasion.

Table 2 shows the analytical results depending on the pH value and active carbon of Series A1. DC is the abbreviation for dry content. OD for the optical density. Feed denotes the solution comprising biomass and oligosaccharides and disaccharide. Permeate is the resulting solution after first membrane filtration, concentrate the remainder of the feed.

TABLE 2

| | | | H₂SO₄-10% | | DC | | 3.2-Di-Fl | 2FL | Lactose | Protein | |
|---|---|---|---|---|---|---|---|---|---|---|---|
| Series | Batch | pH | [g/kg] | Sample | [%] | OD | [g/l] | [g/l] | [g/l] | [g/l] | APHA |
| A1 with 50 nm | Batch 1 | 7.0 | 0 | Feed | 15.9 | 160 | 1.14 | 47.21 | 9.31 | 4.51 | |
| Al2O3 membrane | | | | Permeate | 5.9 | | 0.84 | 29.13 | 5.68 | 1.46 | 8116 |
| | | | | Concentrate | 19.0 | 307 | 0.78 | 24.7 | 1.23 | 5.81 | |
| | | 5.0 | 26.3 | Feed | 16.1 | 242 | 1.14 | 47.21 | 9.31 | 4.51 | |
| | | | | Permeate | 5.69 | | 0.78 | 28.88 | 5.67 | 0.32 | 7952 |
| | | | | Concentrate | 18.9 | 237 | | | | 5.74 | |
| | | 4.0 | 41.7 | Feed | 15.9 | 160 | 1.14 | 47.21 | 9.31 | 1.31 | |
| | | | | Permeate | 5.9 | | 0.77 | 29.21 | 5.61 | 0.19 | 7854 |
| | | | | Concentrate | 19 | 307 | 0.7 | 24.51 | 4.66 | 1.38 | |
| | | 3.5 | 53.8 | Feed | 15.9 | 151 | 1.14 | 47.21 | 9.31 | 4.51 | |
| | | | | Permeate | 5.91 | | 0.82 | 28.99 | 5.62 | 0.14 | 7814 |
| | | | | Concentrate | 17.7 | 293 | 0.74 | 24.83 | | 0.53 | |

The following results are derivable from table 2:

A variation of the pH value has no influence on the color value of the permeate. Lower APHA values at lower pH values are the result of a minor dilution of the fermentation broth by 10% sulfuric acid. The concentration of protein is significantly reduced at lower pH value. The pH value of the fermentation broth has no significant influence on the oligosaccharides 3.2-Di-fucosyllactose (3.2-Di-Fl) and 2′Fucosyllactose (2FL) or the disaccharide lactose.

Table 3 shows the analytical results depending on the pH value and active carbon of Series A2. DC is the abbreviation for dry content. OD for the optical density.

TABLE 3

| | | | | DC | | | 3.2-Di-Fl | 2FL | 2F-Lactulose | Lactose | Protein |
|---|---|---|---|---|---|---|---|---|---|---|---|
| Series | Batch | pH | Sample | [%] | APHA | OD | [g/l] | [g/l] | [g/l] | [g/l] | [g/l] |
| A2 with 50 nm | Batch 2 | 7.0 | Feed | 17.8 | | 138 | 3.43 | 62.07 | 0.6 | 4.28 | 0.478 |
| Al2O3 membrane | | | Permeate | 7.61 | 4196 | | 1.99 | 34.54 | 0.43 | 2.54 | 0.124 |
| | | | Concentrate | 18.5 | | 136 | | | | | 1.882 |
| | | 7.0 + 1% AC | Feed | 18.3 | | 119 | 3.29 | 62.22 | 0.33 | 3.93 | 0.964 |
| | | | Permeate | 7.9 | 1467 | | 2.14 | 37.69 | 0.26 | 2.25 | 0.073 |
| | | | Concentrate | 17.3 | | 237 | 1.89 | 31.39 | 0.54 | 0.12 | 1.41 |
| | | 4.0 | Feed | 17.3 | | 150 | 2.83 | 54.98 | 0.59 | 0.89 | 0.76 |
| | | | Permeate | 8.2 | 4784 | | 1.90 | 35.00 | 0.37 | 2.57 | 0.019 |
| | | | Concentrate | 17.7 | | 434 | | | | | 0.026 |
| | | 4.0 + 1% AC | Feed | 16.8 | | 151 | 2.83 | 55.52 | 0.34 | 3.65 | 0.760 |
| | | | Permeate | 7.8 | 781 | | 1.73 | 33.66 | 0.27 | 2.26 | 0.019 |
| | | | Concentrate | 18.3 | | 293 | 1.61 | 29.14 | 0.33 | 2.38 | 0.026 |

The following results are derivable from table 3:

Adding 1% active carbon to the fermentation broth reduces the color value of the permeate. At a pH value of 7. 1% active carbon reduces the color value at approximately 65%. At a pH value of 4.1% active carbon reduces the color value at approximately 84%. Thus. the color value is below the upper end of 1000 and a further decolorization is not necessary. Adding active carbon at a pH value of 7 reduces the concentration of protein within the permeate at approximately 40%. whereas no effect in this respect by adding active carbon can be derived at a pH value of 4 over the pH effect on protein concentration. Nevertheless. the concentration of protein within the permeate at a pH value of 4 and with adding 1% active carbon is smaller by a factor of 4 if compared to the concentration of protein within the permeate at a pH value of 7 and with adding of 1% active carbon. Adding active carbon has no significant influence on the concentration of the oligosaccharides 3.2-Di-fucosyllactose (3.2-Di-Fl). 2′Fucosyllactulose (2F-Lactulose) and 2′Fucosyllactose (2FL). within the permeate at both pH values. Thus. it can be derived that these components do not adhere to the active carbon in significant amounts. The disaccharide lactose shows in this experiment a small reduction in concentration when active carbon is used. yet the beneficial effect of lowered pH and active carbon allow for the application of the inventive method for this disaccharide.

Table 4 shows the analytical results depending on the pH value and active carbon of Series A3. DC is the abbreviation for dry content. OD for the optical density.

| | | AC | | | DC | | | 3.2-Di-Fl | 2FL | 2F-Lactulose | Lactose | Protein |
|---|---|---|---|---|---|---|---|---|---|---|---|---|
| Series | Batch | [%] | [h] | Sample | [%] | APHA | OD | [g/l] | [g/l] | [g/l] | [g/l] | [g/l] |
| A3 | Batch 3,. pH = 4 | 0 | 0 | Feed | 16.5 | | 218 | 1.60 | 43.40 | 0.16 | 1.28 | 0.050 |
| | | | | Permeate | 7.3 | 4815 | | 0.97 | 26.32 | 0.10 | 0.81 | 0.019 |
| | | | | Concentrate | 15.9 | | 370 | | | | | 0.026 |
| | | 1 | 24 | Feed | 16.5 | | 229 | 1.54 | 41.73 | 0.15 | 1.23 | 0.050 |
| | | | | Permeate | 7.3 | 772 | | 1.00 | 26.90 | | 1.2 | 0.001 |
| | | | | Concentrate | 20.4 | | | 1.06 | 23.79 | 0.1 | 0.79 | 0.001 |
| | | 2 | 24 | Feed | 16.7 | | | 1.46 | 37.35 | na | 1.47 | 0.050 |
| | | | | Permeate | 6.9 | 247 | | 1.01 | 25.78 | na | 1.16 | 0.001 |
| | | | | Concentrate | 21.7 | | | 1.03 | 24.03 | 0.13 | 0.81 | 0.001 |
| | | 1 | 0.3 | Feed | 16.5 | | | 1.69 | 42.43 | na | 1.37 | 0.050 |
| | | | | Permeate | 7.1 | 795 | | 1.09 | 27.50 | na | 0.94 | 0.002 |
| | | | | Concentrate | 19.2 | | | 0.97 | 24.7 | na | 0.89 | 0.000 |
| | | 2 | 0.3 | Feed | 17.2 | | 251 | 1.54 | 39.41 | na | 1.37 | 0.050 |
| | | | | Permeate | 7.1 | 320 | | 1.11 | 27.70 | na | 0.93 | 0.000 |
| | | | | Concentrate | 21.6 | | | 1.01 | 25.44 | na | 0.86 | 0.000 |
| | | 1 | 3 | Feed | 17.0 | | | 1.65 | 42.93 | na | 1.40 | 0.050 |
| | | | | Permeate | 7.4 | 700 | | 1.04 | 26.53 | na | 1.03 | 0.002 |
| | | | | Concentrate | 18.6 | | | 0.94 | 24.07 | na | 0.85 | 0.001 |

The following results are derivable from table 4:

Adding active carbon reduces the concentration of protein within the permeate at 95% if compared to the experiment without adding active carbon. An increase of the added amount of active carbon from 1% to 2% has no significant or detectable influence on the concentration of protein. Adding active carbon significantly reduces APHA. The reduction is approximately 85% with adding 1% active carbon and is 93% to 95% with adding 2% active carbon. a Longer adsorption time before filtration has no significant or detectable influence on APHA or the concentration of protein. Neither a prolonged adsorption time nor an increase in the amount of active carbon used had a strong effect on the oligosaccharides 3.2-Di-fucosyllactose (3.2-Di-Fl). 2′Fucosyllactulose (2F-Lactulose) and 2′Fucosyllactose (2FL) or the disaccharide lactose in the permeate.

Additional tests showed that these good results could be further improved when a second diafiltration step was used.

In another experiment (Series A4) the effects of adding the active carbon before or after the pH lowering of the solution comprising biomass (also called feed) were tested. In this experiment, only the first diafiltration step was used with a DF of 3, the first membrane filtration was stopped and the permeate and the concentrated solution comprising the biomass were analysed and the two treatments compared.

Table 5 shows the analytical results depending on the pH value and active carbon of Series A4.

TABLE 5

| | | | | DC | | | 3.2-Di-Fl | 2FL | 2F-Lactulose | Lactose | Protein |
|---|---|---|---|---|---|---|---|---|---|---|---|
| Series | Batch | pH | Sample | [%] | APHA | OD | [g/l] | [g/l] | [g/l] | [g/l] | [g/l] |
| A4 | Batch 4 | 4.0 then | Feed | 17.3 | | 146 | 1.61 | 50.79 | 0.20 | 1.11 | 0.133 |
| | | 1% AC | Permeate. | 5.9 | 1386 | | 1.15 | 30.91 | | 0.71 | 0.01 |
| | | | Concentrate | 15.9 | | | 0.70 | 16.88 | 0.10 | 0.22 | 0.079 |
| | | 1% AC | Feed | 16 8 | | 146 | 1 62 | 51.47 | 0.16 | 0.75 | 0.137 |
| | | then | Permeate | 6.0 | 1555 | | 1.13 | 29.06 | 0.12 | 0.51 | 0.031 |
| | | pH 4 | Concentrate | 14.4 | | | 0.58 | 14.24 | 0.04 | 0.21 | 0.610 |

The following results are derivable from table 5:

If the pH value is adjusted to 4 first and then adding active carbon reduction of protein and color is better at approximately 15% than if the active carbon is added first and then the pH value is adjusted. No strong effects on the measured oligosaccharides were observed. A slightly stronger retention of the disaccharide lactose was seen when the pH value was lowered first. Empty cells indicate that a value was not determined.

A more stable performance of the membrane (Flux. TMP. DP) was obtained when the pH value was adjusted to 4 first and then the active carbon suspension was added than in an opposite way.

### Example 2

Hereinafter only differences from Example 1 will be described and identical parameters are not repeated.

A fermentation broth as a complex solution comprising biomass and at least one oligosaccharide has been prepared. The pH value thereof has been lowered to 4±0.1 by means of adding 38 g 20% sulfuric acid per kg fermentation broth. Further. 1% active carbon powder has been added. The separation was carried out with a hydrophilic 50 kDa polyethersulfone (PES) membrane (NADIR® UH050 P. MICRODYN-NADIR GmbH. Kasteler Straβe 45. 65203 Wiesbaden. Germany).

Table 6 shows performance data of a biomass separation as a function of adsorption time at pH=4 with 1% active carbon powder and cross-flow speeds for different fermentation broths. “Pe” denote the permeate amount.

TABLE 6

| | | AC | | | Flux | | | | cross |
|---|---|---|---|---|---|---|---|---|---|
| | | | ads. | Pe | [kg/ | TMP | DP | Temp. | flow |
| Batch | pH | [%] | [h] | [kg] | m²h] | [bar] | [bar] | [° C.] | [m/s] |
| Batch 4 | 4. 0 | 1.0 | 0.3 | 3.77 | 23 | 1.51 | 2.03 | 39.5 | 1.2 |
| | | 1.0 | 0.3 | 4.02 | 21 | 1.67 | 2.58 | 39.3 | 1.1 |
| Batch 5 | 4. 0 | 1.0 | 1.0 | 9.00 | 45 | 1.90 | 2.46 | 37.6 | 1.5 |
| Batch 6 | | 1.0 | 1.0 | 6.83 | 35 | 1.94 | 2.12 | 39.0 | 1.7 |
| Batch 7 | 4.0 | 1.0 | 1.0 | 4.47 | 12 | 1.2 | 1.18 | 38.2 | 1.0 |

The following results are derivable from table 6:

Using different starting solutions, pH4.4, 1% active carbon and low cross-flow speeds were tested and resulted in good membrane performance. Longer incubation periods of the adsorbing agent in the starting solution were not required.

Comparing the results from tables 2, 3 and 6, it can be concluded that at pH=4 and with 1% active carbon the membrane performance was higher by a factor 4 to 10 in comparison with performance measured with fermentation broth without active carbon and at pH value of 7 and that the reduction of the cross-flow speed from 1.5 m/s to 1.1 m/s results in significant flux reduction but the performance is still higher by a factor 2 in comparison to the trials at a pH value of 7 with a cross-flow speed of 1.5 m/s.

In a further experiment the batches of fermentation broth shown in table 6 were used as starting solution experiments for further tests. Batch 4 was used to test the two variants of first setting pH to 4.0 and then adding 1% w/w active carbon, or first a adding the same percentage of carbon and afterwards setting the pH to 4.0, followed by the first membrane filtration. As observed before, the permeate of the membrane filtration showed 3 to 4 times more protein when the pH was set after the addition of active carbon compared to adding the active carbon to a solution already at pH 4.0. Also, the APHA values of the permeate were higher when active carbon was added before the pH adjustment. Both treatments had similar effects on the oligosaccharides and disaccharides measured, these were largely recovered in the permeate and losses in the retentate were in the order of 10% to 20%, in some cases up to 30%.

Tests with batches 5, 6 and 7 and setting pH to 4.0 and then adding 1% w/w active carbon, confirmed the permeate to contain as little as 5 to 15% of the protein amount of the starting solution. APHA values of the permeate below 700 (batch 7) and below 400 (batches 5 and 6) were achieved. With respect to the oligosaccharides and disaccharides, again the large majority was recovered in the permeate, with the retentate containing amounts similar to those observed for batch 4.

Additional observations were that oligosaccharide and lactose concentration in fermentation broth may vary significantly. yet the inventive methods can be applied with similar results on the oligosaccharides and lactose nonetheless; and a lower color number in the permeate as a trend correlates with a lower the protein concentration in the permeate.

In addition, several batches of fermentation broths produced with standard methods comprising 6′-sialyllactose or Lacto-N-tetraose, have been tested in the inventive methods. The results when the pH was lowered first, and then active carbon was added were comparable and often even better than those shown above. For example, fermentation broths comprising Lacto-N-tetraose starting with a high concentration of color components resulting in APHA values of 7000 or more in the feed, gave permeates after the first membrane filtration with an APHA value of below 1000, but typically below 300 and even as low as below 100. The protein concentration was lowered from typically around 3 g/l to less than 0.01 g/l. The vast majority, typically above 95%, of the Lacto-N-tetraose originally found in the fermentation broth was present in the combined permeate. Similarly, for other oligosaccharides present and also for the disaccharide lactose most was present in the combined permeate and only minor amounts found in the retentate at the end of the first membrane filtration. The applied DF values were below 3.

Also, fermentation broths comprising 6′-sialyllactose with APHA values of around 7000, after said first membrane filtration resulted in permeates with an APHA value of below 300 and even as low as below 70. The protein concentration was lowered by a factor of 10 or more compared to the starting value in the fermentation broth, at DF values below 3. The vast majority, typically above 90% of the 6′-sialyllactose originally found in the fermentation broth was present in the combined permeate. Similarly, for other oligosaccharides present and also for the disaccharide lactose most was present in the combined permeate and only minor amounts found in the retentate at the end of the first membrane filtration.

It was also found that performing the methods with a pH of below 5.5 improved flux in the first membrane filtration compared to higher pH values (cross-flow speed 3.5 m/s, temperature 30° C.; DF=3). This improved even further when the pH value of the solution comprising the biomass and the 6′-sialyllactose was pH 4.2. Compared to pH 6.3, the flux more than doubled when pH 5.2 was used and tripled when the pH value was pH 4.2.

### CITED LITERATURE

WO 2015/032412
EP 2 379 708
CN 100 549 019
EP 2 896 628
U.S. Pat. No. 9,944,965

1.-15. (canceled)

16. A method for separating biomass from a solution comprising biomass and at least one oligosaccharide, comprising:
providing the solution comprising biomass and oligosaccharides;
setting the pH value of the solution below 7 by adding at least one acid to the solution comprising biomass and the at least one oligosaccharide;
adding at least one adsorbing agent to the solution comprising biomass and oligosaccharides; and
carrying out a first membrane filtration, so as to separate the biomass from the solution comprising at least one oligosaccharide.

17. The method according to claim 16, wherein the pH value of the solution is lowered to a pH value in the range of 3.0 to 5.5.

18. The method according to claim 16, wherein said at least one acid is an acid selected from the group consisting of H₂SO₄, H₃PO₄, HCl, HNO₃ and CH₃CO₂H.

19. The method according to claim 16, wherein said adsorbing agent is added in an amount in the range of 0.5% to 3% by weight.

20. The method according to claim 16, wherein said adsorbing agent is added as a powder having a particle size distribution with a diameter d50 in the range of 2 μm to 25 μm.

21. The method according to claim 20 wherein said adsorbing agent is added as a suspension of the powder in water.

22. The method according to claim 16, wherein said first membrane filtration is carried out as cross-flow microfiltration or cross-flow ultrafiltration.

23. The method according to claim 22, wherein said cross-flow microfiltration or cross-flow ultrafiltration includes a cross-flow speed in the range of 0.5 m/s to 6.0 m/s.

24. The method according to claim 22, wherein said cross-flow speed is equal to or below 3 m/s.

25. The method according to claim 16, wherein said first membrane filtration is carried out at a temperature of the solution in the range of 8° C. to 55° C.

26. The method according to claim 16, wherein said first membrane filtration is carried out by means of a ceramic microfiltration or ultrafiltration membrane having a pore size in the range of 20 nm to 800 nm, or wherein said first membrane filtration is carried out by means of a polymeric microfiltration membrane or polymeric ultrafiltration membrane having a cut-off in the range of 10 kDa to 200 nm.

27. The method according to claim 16, further comprising carrying out a second membrane filtration with the solution comprising oligosaccharides obtained by the first membrane filtration.

28. The method according to claim 27, wherein said second membrane filtration is an ultrafiltration and is carried out by means of an ultrafiltration membrane having a cut-off in the range of 1.5 kDa to 10 kDa.

29. The method according to claim 27, wherein said second membrane filtration is carried out at a temperature of the solution being in the range of 5° C. to 15° C.

30. The method according to claim 16, wherein said at least one oligosaccharide comprises human milk oligosaccharide.

31. The method according to claim 16, wherein the adsorbing agent comprises active carbon.

32. The method according to claim 16, wherein the pH value of the solution is lowered to a range from 4.0 to 4.5.

33. The method according to claim 16, wherein the adsorbing agent is added in an amount in the range of 1.0% to 2.0% by weight and the adsorbing is added as a powder having a particle size distribution with a diameter d50 in the range of 3 μm to 7 μm.

34. The method according to claim 16, wherein the first membrane filtration comprises microfiltration or ultrafiltration and is carried out at a temperature of the solution in the range of 30° C. to 40° C.

35. The method according to claim 16, wherein the at least one oligosaccharide comprises at least one of 2′-fucosyllactose, 6′-sialyllactose and Lacto-N-tetraose.
